(19) United States
(12) Patent Application Publication
(10) Pub. No.: US 2024/0382671 A1
(43) Pub. Date: Nov. 21, 2024

(54) INFUSION DEVICE HAVING A PROCESSING DEVICE CONFIGURED TO DETERMINE A VALUE INDICATIVE OF A SENSITIVITY OF A SENSOR DEVICE

(71) Applicant: FRESENIUS VIAL SAS, Brézins (FR)

(72) Inventor: Rémy Wolff, Tullins (FR)

(21) Appl. No.: 18/683,768

(22) PCT Filed: Sep. 7, 2022
(86) PCT No.: PCT/EP2022/074885

§ 371 Date: Feb. 14, 2024

(30) Foreign Application Priority Data
Sep. 10, 2021 (EP) 21315156.6

(51) Int. Cl. A61M 5/142

(52) U.S. Cl. CPC A61M 5/14212; A61M 2205/332

(57) ABSTRACT

An infusion device (1) for administering a medical fluid to a patient (P) comprises a receptacle (12) for receiving a syringe (2). the syringe (2) comprising a tube (20) and a piston (21) movable with respect to the tube (20), a pusher device (11) for exerting a force onto the piston (21) for delivering a medical fluid from the tube (20) towards a patient (P). A sensor device (14) is arranged on the pusher device (11) for measuring a force exerted on the piston (21) by the pusher device (11). A holding element (17) is arranged on the pusher device (11), the holding element (11) being movable with respect to the pusher device (11) between a non-actuated position, in which the holding element is configured to hold the piston (21) on the pusher device (11), and an actuated position for releasing the piston (21) from the pusher device (11). A processing device (15) serves for controlling operation of the infusion device (1). Herein, the processing device (15) is configured to evaluate a sensor signal measured by the sensor device (14) in the actuated position of the holding element (17) and to determine a value indicative of a sensitivity of the sensor device (14) based on said sensor signal measured by the sensor device (14) in the actuated position of the holding element (17) and based on a reference value (S0).

The invention relates to an infusion device for administering a medical fluid to a patient according to the preamble of claim 1 and to a method for operating an infusion device.

An infusion device of the type concerned herein comprises a receptacle in which a syringe having a tube containing a medical fluid to be administered to a patient and a piston for pushing the medical fluid out of the tube can be received. The syringe infusion device comprises a pusher device for acting onto the piston of the syringe for pushing the piston into the tube in order to deliver the medical fluid from the tube towards the patient. A sensor device in the shape of a force sensor is placed on the pusher device for measuring the force exerted onto the piston and to derive, from the force measurement, the pressure within the tube and an infusion line connected to the tube. A processing device serves to control operation of the infusion device.

Herein, a holding element is arranged on the pusher device, the holding element being movable with respect to the pusher device between a non-actuated position, in which the holding element is configured to hold the piston on the pusher device, and an actuated position for installing or releasing the piston from the pusher device. By means of the holding element a piston of a syringe received in the receptacle of the infusion device can operatively be connected to the pusher device, such that the piston can be moved together with the pusher device for operating the infusion device.

An infusion device as concerned herein may for example have a mechanical setup as it is described in EP 2 686 039 B1.

The force measurement serves, in general, to monitor an infusion operation and, in particular, to detect an occlusion in an infusion line. If an occlusion in the infusion line connected to the patient is present, the pressure in the infusion line will rise, which can be observed by the force measurements of the sensor device. If the pressure rises beyond a predefined threshold, it is concluded that an occlusion is present, such that appropriate countermeasures can be taken for releasing the occlusion or for interrupting the infusion operation.

During an infusion operation, an occlusion must reliably be detected. In turn, false alarms are to the avoided to prevent an alarm fatigue of a user. Hence, reliable force measurements allowing for an accurate estimation of the pressure within the infusion line are required.

For example, a load cell comprising for example strain gauges is used as sensor device. A load cell of this kind may for example comprise a sensor support element for example made from aluminum and a strain gauge arrangement, such as a Wheatstone bridge circuit, fixed on the sensor support element. If a force is exerted on the load cell, a (bending) deformation of the sensor support element will cause an electric signal within the strain gauge arrangement placed on the sensor support element, such electric signal being proportional to the force exerted on the load cell and hence allowing for a force measurement.

In a load cell a drift may occur, for example caused by a temperature variation or by an aging of the load cell, the drift possibly influencing the sensor signal provided by the load cell and thus potentially having an impact on the accuracy of the force measurement. Herein, two different drift effects may be present. Within the so-called zero drift the output sensor signal when no force is applied to the sensor device varies, for example over time and/or over temperature. Within the so-called span drift, in contrast, the output sensor signal when a force is applied to the sensor device may change over time and/or over temperature. Both effects may have an impact on the accuracy of the force measurement. It hence is desirous to identify drift effects during operation of the infusion device in order to allow for a reliable and accurate operation of the infusion device.

EP 1 267 960 B1 discloses an infusion pump system with an occlusion detection system.

US 2014/0163522 A1 discloses an infusion device with a sensor for detecting a drift.

It is an object of the instant invention to provide an infusion device and a method for operating an infusion device which allow for a reliable operation, in particular a reliable sensing of sensor signals indicative of a force exerted on a piston of a syringe.

The object is achieved by means of an infusion device comprising the features of claim 1.

Accordingly, the processing device is configured to evaluate a sensor signal measured by the sensor device in the actuated position of the holding element and to determine a value indicative of a sensitivity of the sensor device based on said sensor signal measured by the sensor device in the actuated position of the holding element and based on a reference value.

The holding element serves to operatively connect a piston of a syringe received in the receptacle of the infusion device to the pusher device. The holding element may be moved between its non-actuated position and an actuated position, wherein the holding element is configured to operatively connect, in the non-actuated position, a piston to the pusher device and to release the piston from the pusher device when it is moved into the actuated position.

When releasing a piston from the pusher device, or for placing and arresting a piston on the pusher device, the holding element is moved to its actuated position. This generally takes place at a defined force acting onto the pusher device and causing a sensor signal at the sensor device, such that a sensor reading in the actuated position of the holding element may be used to calibrate the sensor device. Namely, if the force acting onto the sensor device in the actuated position of the holding element is known, the sensor signal as measured in the actuated position of the holding element can be associated with the known force, such that a sensor sensitivity can be determined and can be used during the subsequent operation of the infusion device.

In particular, by determining a value indicative of a sensitivity of the sensor device and by monitoring such value of the sensitivity of the sensor device during operation of the infusion device, a drift can be identified, such that countermeasures may be taken once it is found that the sensor readings may have become unreliable due to a drift in the sensor device.

By using a calibration taking a sensor signal in the actuated position of the holding element into account, a simple and effective way of calibrating the sensor device is provided, allowing for a reliable and computationally efficient possibility to monitor for a potential drift in sensor readings of the sensor device.

In one embodiment, the sensor device comprises a sensor support element configured to react to a force exerted on the piston by the pusher device, wherein the holding element is mounted on the sensor support element and is movable with respect to the sensor support element between the non-actuated position and the actuated position. The sensor device in particular may be configured as a so-called load cell, the sensor support element being elastically deformable in reaction to a force acting onto the sensor support element. One or multiple sensor elements for example in the shape of strain gauges or extension gauges may be placed on the sensor support element, such that a deformation of the sensor support element due to a force acting onto the sensor support element can be picked up by the arrangement of sensor elements and may be used to output a sensing signal indicative of a force acting onto the sensor device.

The sensor support element may for example have the shape of a metal body, e.g. made from aluminum, and may be deformable in reaction to a force acting onto the sensor support element, in particular caused by the pusher device acting onto the piston for moving the piston of the syringe with respect to the tube for delivering a medical fluid from the syringe towards a patient. Sensor elements herein may be arranged on the sensor support element and may electrically be connected e.g. to form a Wheatstone bridge circuit, the Wheatstone bridge circuit being operative to sense a bending deformation of the sensor support element in reaction to a force acting onto the sensor support element. An electrical signal obtained using the sensor element arrangement may be proportional to the force exerted on the sensor device, such that a force measurement using the sensor device becomes possible.

The holding element, advantageously, is arranged on the sensor support element and is movable with respect to the sensor support element in order to move the holding element between its non-actuated position and its actuated position. Herein, in one embodiment a spring element elastically pre-tensions the holding element with respect to the sensor support element along a direction of motion along which the holding element is movable with respect to the sensor support element in order to move the holding element between the non-actuated position and the actuated position. The spring element in particular causes a spring elastic tensioning which, due to the spring constant of the spring element, increases when the holding element is moved along the direction of motion from the non-actuated position to the actuated position. If the spring constant of the spring element is known, and if further the displacement of the holding element when moving the holding element from the non-actuated position to the actuated position is known, the force caused by the holding element on the sensor support element is known, such that a sensor reading of the sensor device in the actuated position of the holding element can be related to a known force, which can be used for calibrating the sensor device.

The holding element in general is movable with respect to the sensor support element along the direction of motion, which may be directed for example along a pushing direction along which the pusher device is movable in order to act onto the piston of the syringe for administering a medical fluid from the syringe towards a patient. By moving the holding element from the non-actuated position to the actuated position, the holding element herein may be removed from the sensor support element along the direction of motion, wherein in addition the holding element may for example be pivoted in order to allow a placement of a piston head of the piston on the pusher device, as it is for example described in EP 2 686 039 B1.

The holding element may be arranged on the sensor device such that a first balance of forces exerted by the holding element on the sensor device is substantially zero or at least close to zero in the non-actuated position of the holding element. In that the holding element is arranged on the sensor support element, and as the spring element pre-tensions the holding element with respect to the sensor support element, the holding element does not cause a net force on the sensor support element when the holding element is in its non-actuated position, in particular in a state in which no syringe is received in the receptacle of the infusion device and hence no piston is operatively connected to the pusher device. In the non-actuated position, the holding element hence does not, by itself, cause a deformation of the sensor support element, such that the sensor device does not pick up any force caused by the holding element on the sensor support element.

This may, in one embodiment, be used to determine the reference value in order to record a so-called zero-reading of the sensor device, i.e., a sensor signal of the sensor device in a state in which no load acts onto the sensor device. In one embodiment, the reference value is determined while the holding element is in the non-actuated position and no piston is operatively connected to the pusher device. In this state a holding arm of the holding element may be caused to be in abutment with the sensor device due to a pre-tensioning of the spring element biasing the holding element with respect to the sensor support element, wherein the balance of forces caused by the holding element on the sensor support element is zero, such that the sensor signal of the sensor device reflects a situation in which no load acts onto the sensor device.

In one embodiment, the holding element is arranged on the sensor device such that a second balance of forces exerted by the holding element on the sensor device in the actuated position is different than zero. In the actuated position the holding element is displaced with respect to the sensor support element, preferably against the spring-elastic force of the spring element tensioning the holding element with respect to the sensor support element. The displacement of the holding element is for example caused by an actuation mechanism by which the holding element is moved on the pusher device, a force introduced into the holding element in this way causing a load on the sensor device and hence a sensor signal associated with that load. If the spring constant of the spring element is known, and if further the displacement between the non-actuated position and the actuated position is known, the force exerted by the holding element on the sensor device can be computed to be

$${F}_{1} = K \cdot {X}_{1} + {F}_{0} .$$

F₁ herein indicates the force acting onto the sensor device in the actuated position of the holding element, K indicates the spring constant of the spring element pre-tensioning the holding element with respect to the sensor support element, X₁ is the displacement by which the holding element is displaced with respect to the sensor support element, and F₀ is an initial tensioning force caused by a tensioning of the holding element with respect to the sensor support element in the non-actuated position (which may be computed from an initial deformation (e.g., compression) of the spring element).

Generally, the processing device may be configured to determine said value indicative of the sensitivity of the sensor device based on a difference between said sensor signal measured by the sensor device in the actuated position of the holding element and the reference value. For computing the value indicative off the sensitivity, hence, the measured value is set in relation to the reference value, in that a difference between the measured value and the reference value is formed. The difference as such may be used as a value indicative of (a change in) the sensitivity. Alternatively, the difference may be modified for computing the value indicative of the sensitivity by taking further parameters into account.

The processing device, in one embodiment, is configured to determine said value indicative of the sensitivity of the sensor device based on the sensor signal measured by the sensor device in the actuated position of the holding element and in addition based on the displacement by which the holding element is displaced with respect to the pusher device in the actuated position with reference to the non-actuated position along a direction of motion. From the displacement the force acting onto the sensor device in the actuated position can be determined using the spring constant of the spring element pre-tensioning the holding element with respect to the sensor support element, such that the force acting onto the sensor support element by means of the holding element in the actuated position is known and can be related to the sensor reading of the sensor device in the actuated position of the holding element.

In particular, the processing device may be configured to determine said value indicative of the sensitivity of the sensor device based on the following equation:

$${K}_{S} = ( {S}_{1} - {S}_{0} ) / {X}_{1}$$

where K_{S} indicates said value indicative of the sensitivity of the sensor device, S₁ indicates the sensor signal measured by the sensor device in the actuated position of the holding element, S₀ indicates a reference value, and X₁ indicates the displacement of the holding element in the actuated position.

In another embodiment, the processing device is configured to determine said value indicative of the sensitivity of the sensor device based on the following equation:

$${{K}_{S}}^{′} = ( {S}_{1} - {S}_{0} )$$

where K_{S}′ indicates said value indicative of the sensitivity of the sensor device, S₁ indicates the sensor signal measured by the sensor device in the actuated position of the holding element, and S₀ indicates the reference value. In this embodiment, hence, a value indicative of the sensitivity is determined without reference to an actual displacement of the holding element, which in a realistic scenario may not always be known. In this embodiment, an initial Ks′ value may be determined during the manufacturing or during initial start-up of the infusion device. The Ks′ value may then repeatedly be determined anew during operation of the infusion device, and based on a deviation of the actual Ks′ value during operation from the initial Ks′ value a relative change in sensitivity may be derived and taken into account, without determining an actual, absolute sensitivity value.

The reference value in particular may be determined as described above, namely in the non-actuated position of the holding element in a state in which no piston is operatively connected to the pusher device, such that the reference value indicates a zero-reading of the sensor device in a state in which no load acts onto the sensor device.

The reference value may in particular be zero or may be close to zero. Although the holding element does not cause a net force on the sensor device, a local deformation of the sensor support element however may cause a non-zero signal value of the sensor device, which is reflected by the reference value.

The determination of the sensor sensitivity may be used for monitoring the sensor device for a potential drift during operation of the infusion device. For this, in a calibration procedure, for example prior to initial operation of the infusion device or repeatedly at certain intervals during operation of the infusion device, a default value for said value indicative of the sensitivity of the sensor device may be determined. Then, subsequently, during operation of the infusion device, in particular in situations in which the holding element is brought into the actuated position for example for placing a syringe on the infusion device or for releasing a syringe from the infusion device, the value indicative of the sensitivity of the sensor device may be repeatedly determined anew, such that the sensitivity is monitored and a potential drift in the sensitivity of the sensor device may be identified.

In particular, for the monitoring a current value of the sensitivity of the sensor device may be compared to the default value as determined and stored during a prior calibration procedure. If it is found that the sensitivity value substantially deviates from the default value, this may indicate a drift in the sensor device's sensitivity.

When a drift is detected, countermeasures may be initiated. For example, it may be displayed to a user that a technical error has occurred and a need for maintenance exists. Alternatively or in addition, based on the monitoring the default value for the sensitivity can be corrected, such that in a further monitoring an updated value for the default value is used.

In one embodiment, the reference value (as determined in a non-loaded situation of the sensor device, while the holding element is in the non-actuated position and no piston is operatively connected to the pusher device) may in itself be used for a monitoring of a drift in the sensor device, due for example to aging or temperature. For example, an initial reference value may be set in a calibration procedure, for example at an initial startup of the infusion device or at certain intervals during operation of the infusion device. If subsequently the reference value is determined anew and is found to deviate from the previously stored initial reference value, this may be indicative of a drift in the sensor device signals, which may be accordingly identified by the processing device of the infusion device.

In one embodiment, the holding element is configured, in an operative state in which the holding element is in the non-actuated position and operatively connects the piston to the pusher device, to bias the piston to abut with the sensor device. When the pusher device is not moved and hence does not exert a force onto the piston, another reference value, denoted as a load reference value, may be determined in this operative state, the load reference value indicating a reference for the sensor device in a state in which a piston is connected to the pusher device (but the infusion device is not operated to move the piston). The load reference value should (as the holding element should not cause a net force on the sensor device in a state in which the pusher device is not operated to move the piston) be close to the reference value determined as described above. The load reference value may for example be used to measure a friction force and may assist in identifying an occlusion situation during operation of the infusion device.

In another aspect, a method for operating an infusion device for administering a medical fluid to a patient comprises: receiving a syringe in a receptacle of the infusion device, the syringe comprising a tube and a piston movable with respect to the tube; exerting, using a pusher device, a force onto the piston for delivering a medical fluid from the tube towards a patient; measuring, using a sensor device arranged on the pusher device, a force exerted on the piston by the pusher device; and controlling operation of the infusion device using a processing device. Herein, the method comprises the further steps of evaluating, using the processing device, a sensor signal measured by the sensor device in an actuated position of a holding element, the holding element being arranged on the pusher device and being movable with respect to the pusher device between a non-actuated position for holding the piston on the pusher device and the actuated position for releasing the piston from the pusher device; and determining, using the processing device, a value indicative of a sensitivity of the sensor device based on said sensor signal measured by the sensor device in the actuated position of the holding element and based on a reference value.

The advantages and advantageous embodiments described above for the infusion device equally apply to the method, such that it shall be referred to the above in this respect.

The steps of evaluating said sensor signal measured by the sensor device in the actuated position of the holding element and of determining said value indicative of the sensitivity of the sensor device may take place initially prior to actual operation of the infusion device, and alternatively or in addition during operation of the infusion device, in particular in any situations in which the holding element is brought to its actuated position, for example when releasing a piston of a syringe from the infusion device or when placing a syringe on the infusion device.

The idea of the invention shall subsequently be described in more detail with reference to the embodiments shown in the figures. Herein:

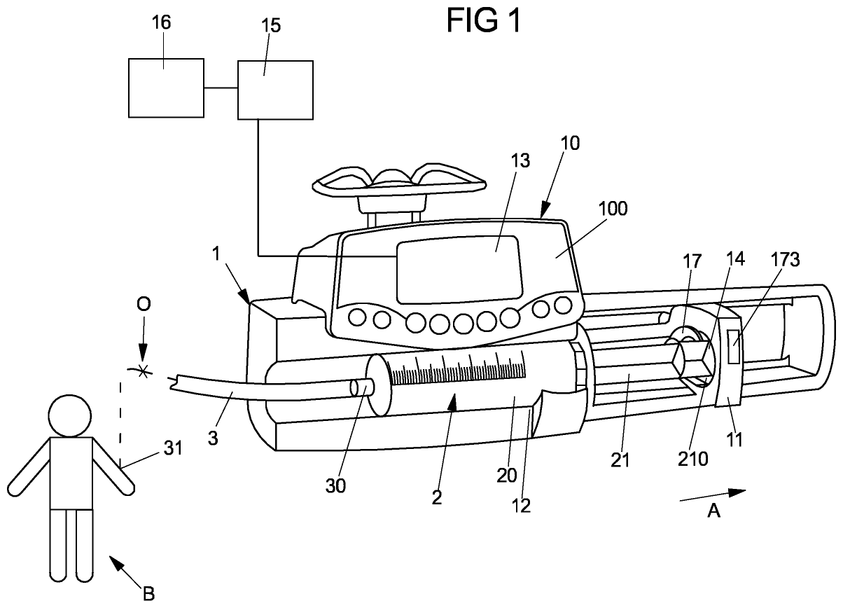

FIG. 1 shows a view of an infusion device configured as a syringe pump;

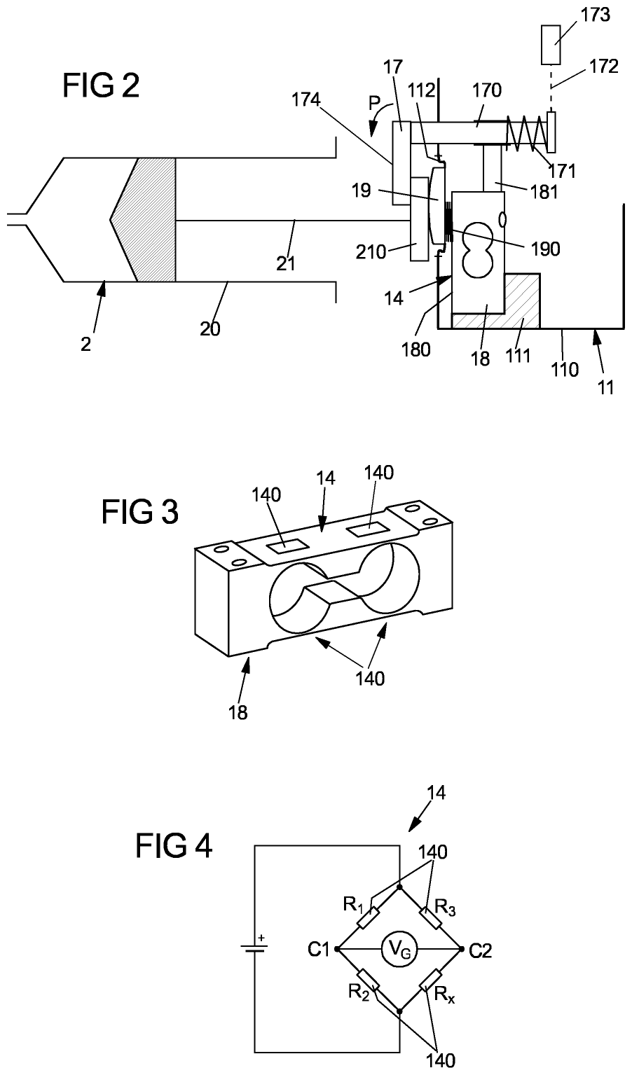

FIG. 2 shows a schematic drawing of a pusher device of the infusion device;

FIG. 3 shows a sensor device in the shape of a load cell;

FIG. 4 shows an electric circuit schematic of the sensor arrangement;

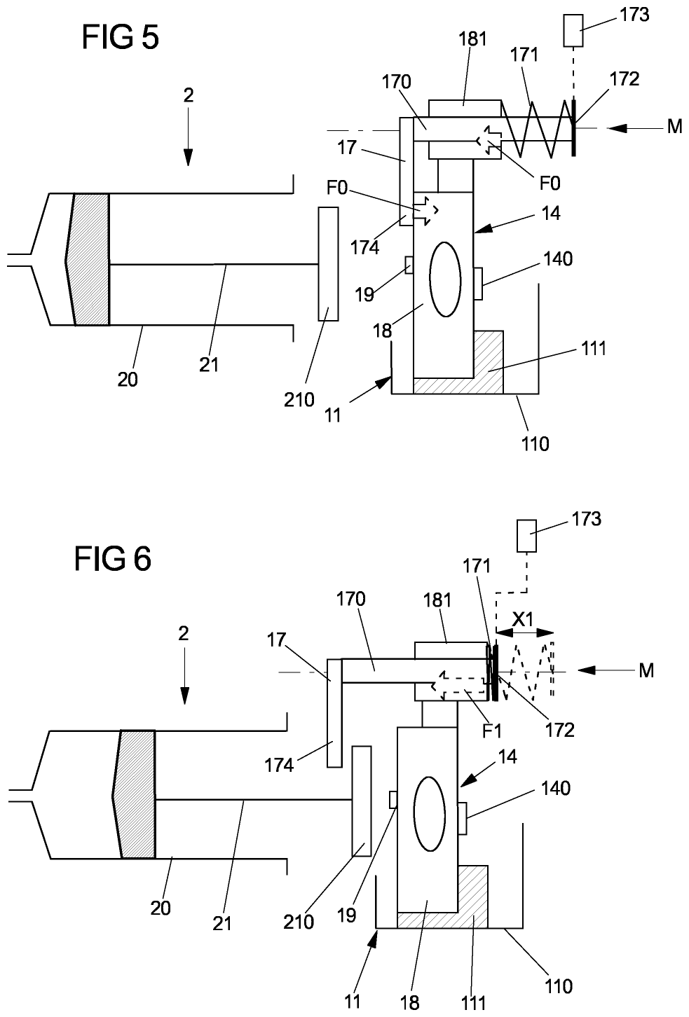

FIG. 5 shows a schematic drawing of a pusher device of an infusion device, in a non-actuated position of a holding element while no piston of a syringe is operatively connected to the pusher device;

FIG. 6 shows a schematic drawing of the arrangement of FIG. 5, with the holding element in an actuated position;

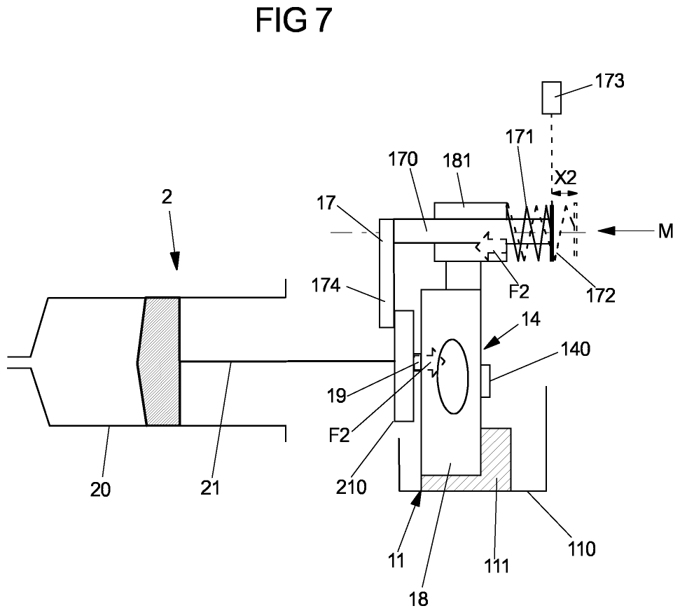

FIG. 7 shows the arrangement of FIG. 6, in a non-actuated position of the holding element while a piston of a syringe is operatively connected to the pushing device; and

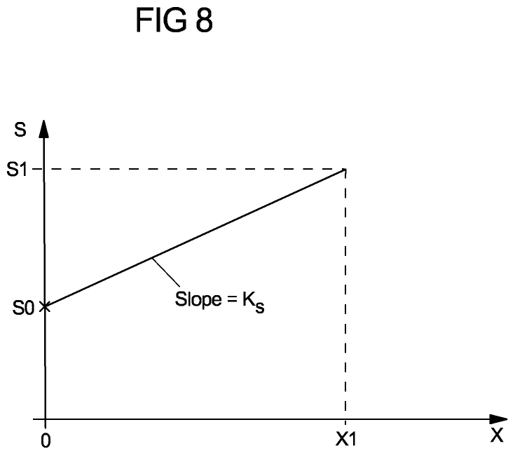

FIG. 8 shows a sensor output of the sensor device as a function of the position of the holding element.

FIG. 1 shows an embodiment of an infusion device 1 in the shape of a syringe pump. The infusion device 1 comprises a housing 10 having a front face 100 and a display device 13 arranged thereon. The display device 13 may for example be a touch-sensitive display allowing a user to enter commands for operation of the infusion device 1 and displaying operational information regarding the process of an actual infusion operation.

The infusion device 1 comprises a receptacle 12 in which a syringe 2 having a (e.g. cylindrical) tube 20 is arranged. A piston 21 is movable within the tube 20 and is in engagement with a pusher device 11 of the infusion device 1. At an end of the tube 20 opposite the piston 21 a delivery line 3 extends from the tube 20 towards a patient B, the delivery line 3 being connected to the tube 20 at a first end 30 and to the patient B at a second end 31.

The piston 21 comprises a head 210 facing away from the tube 20 and being in abutment, in an operatively connected state, with the pusher device 11 of the infusion device 1. During operation of the infusion device 1, the pusher device 11 is typically driven by an electric motor in a pushing direction A such that the piston 21 is moved into the tube 20 and a medical fluid contained in the tube 20 is delivered via the delivery line 3 towards the patient B.

The infusion device 1 comprises a processing device 15 and a storage device 16. Via the processing device 15 the infusion operation of the infusion device 1 is controlled. In the storage device 16 operational parameters, such as mechanical characteristics of the syringe 2 used on the infusion device 1 as well as operational data, may be stored.

During an infusion process a medical fluid, for example a medication or a nutritional fluid for the parenteral feeding of a patient or the like, is delivered from the tube 20 via the delivery line 3 towards the patient B. For this, the piston 21 in operation of the infusion device 1 is continuously pushed into the tube 20 in the pushing direction A such that a desired flow rate is obtained, which is programmed by a user prior to the start of the infusion operation.

The delivery line 3 generally is made of a flexible tubing made for example from a PVC material. The delivery line 3 extends from the tube 20 to the patient B and is, at its first end 30, in fluid connection with the tube 20 and, at its second end 31, for example connected to a needle for providing an intravenous access to the patient B. During an infusion process an occlusion O in the delivery line 3 must be avoided and, if it nevertheless occurs, must be detected such that appropriate countermeasures to overcome the occlusion O can be taken. For this, a force sensor 14 is placed on the pusher device 11 facing the head 210 of the piston 214 measuring a force exerted on the piston 21 during an infusion process. From a force measured by means of the force sensor 14 an estimate of the pressure within the syringe 2 can be obtained, such that the pressure within the syringe 2 and the delivery line 3 can be monitored. If it is found that the pressure within the syringe 2 and the delivery line 3 rises beyond a permissible threshold value, an alarm is triggered indicating that an occlusion O may be present in the system.

Generally, the pressure in the delivery line 3 is very small (almost 0) during normal infusion operation in case no occlusion O is present. If an occlusion O occurs, the pressure will start to rise and will continue to rise (if the occlusion O does not disappear) until a threshold value is exceeded, at which moment an alarm is triggered by the processing device 15 such that a user is warned of the occlusion O.

To observe the pressure in the delivery line 3, the force applied to the piston head 210 of the piston 21 by means of the pusher device 11 is measured by the sensor 14. The force measured in this way allows for an indirect measurement of the pressure within the tube 20, which generally equals the pressure in the delivery line 3.

In particular, the pressure in the tube 20 relates to the measured force. By determining the pressure from the measured force and by comparing the determined pressure to a predefined threshold it can then be concluded whether an occlusion O is present in the delivery line 3 or not. In particular, if it is found that the pressure rises above the threshold, it is concluded that an occlusion O is present.

FIG. 2 shows, in a schematic drawing, the mechanics of an embodiment of a pusher device 11 of the infusion device 1. The pusher device 11 comprises a housing 110 and is movable along the pushing direction A during an infusion operation to push the piston 21 at a constant speed into the tube 20 of the syringe 2 in order to deliver a medical fluid from the tube 20 at a constant dose rate towards the patient B. The pusher device 11 herein is driven by a suitable driving mechanism comprising an electric drive (not shown) controlled by the processing device 15.

For pushing the piston 21 into the tube 20, the piston 21 via its piston head 210 is operatively connected to the pusher device 11 via a holding arm 174 of a holding element 17 mounted on the pusher device 11. The holding element 17 is pivotably mounted via a shaft 170 on a sensor support element 18 of the sensor device 14. The shaft 170, for this, is mounted on a support member 181 integrally connected with the sensor support element 18 such that the shaft 170 is pivotable with respect to the support member 181, and in addition is axially displaceable along a longitudinal direction of motion M, corresponding to the pivoting axis of the holding element 17.

The shaft 170 is pre-tensioned with respect to the support member 181 via a spring element 171 providing a spring elastic force axially on the shaft 170. The holding element 17 herein is movable from a between a non-actuated position, in which the holding element 17 is configured to arrest the piston 21 with its head 210 on the pusher device 11, and an actuated position in which the piston 21 can be released from the pusher device 11, or can be installed on the pusher device 11.

In the non-actuated position the holding element 17 exerts a force axially onto the piston head 210 along the pivoting axis, caused by the spring element 171, in order to bias the piston head 210 into abutment with a pressure transmitting element 19 which is elastically supported, via a spring element 190, on the sensor support element 18 of the sensor device 14 and acts onto the sensor device 14 to transmit a pressure towards the sensor device 14.

The pressure transmitting element 19 is sealed with respect to the housing 110 of the pusher device 11 by means of a sealing membrane 112 extending from the pressure transmitting element 19 and surrounding the pressure transmitting element 19. The inside of the housing 110 of the pusher device 11 hence is closed towards the outside to prevent entrance of moisture and dirt.

The piston 21 is, via the pressure transmitting element 19, in operative abutment on the sensor device 14 such that the sensor device 14 may measure a force exerted on the piston head 210 of the piston 21 by means of the pusher device 11. The sensor support element 18 is mounted within the housing 110 a means of a mounting element 111 such that the sensor support element 18 is fixedly connected to the housing 110 of the pusher device 11.

The mechanics of the infusion device 1 may for example be embodied as it is described in EP 2 686 039 B1.

Via the force sensor 14 the force acting onto the piston 21 is measured, thus allowing for estimating the pressure within the tube 20 and within the delivery line 3, such that an occlusion in the delivery line 3 can be detected by observing the pressure. The sensor device 14, in the illustrated embodiment, has the shape of a load cell, the sensor support element 18 being formed by an integral metal body made for example from aluminum on which an arrangement of sensor elements 140 is placed, as depicted in FIG. 3. The sensor elements 140 in the shape of strain gauges or extension gauges may be electrically connected, as shown in FIG. 4, to form a Wheatstone bridge having nodes C1, C2 in between which an electric voltage signal can be obtained, the voltage signal being proportional to the force exerted on the sensor device 14.

When a force is exerted on the sensor device 14, the sensor support element 18 will be elastically deformed, which will lead to a stretching of some of the sensor elements 140 and a contracting of other sensor elements 140. Such stretching/contracting causes a differential voltage signal in between the nodes C1, C2, which can be picked up and can be used to derive a force measurement.

Within a load cell, a drift may occur, caused by a varying temperature or by aging effects over the lifetime of the sensor device 14. Such drift may have an effect on the accuracy of a force measurement, such that a drift should be detected and potentially be corrected.

Referring now to FIG. 5, the holding element 17 is mounted on a support member 181 fixedly connected to the sensor support element 18, such that the holding element 17 is supported on the sensor support element 18. The holding element 17 herein is axially movable along a direction of motion M with respect to the support member 181, the holding element 17 with its holding arm 174 in addition being rotatable in order to release a piston head 210 from the pusher device 11 or to be able to install a piston 21 with its piston head 210 on the pusher device 11.

The holding element 17 is operatively connected to an actuation mechanism 173 actuatable by a user for actuating the holding element 17 (as it is for example described in EP 2 686 039 B1).

In its non-actuated position, as shown in FIG. 5, and in a state in which no piston 21 of a syringe 2 is installed on the pusher device 11, the holding arm 174 of the holding device 17 abuts the sensor support element 18, wherein the holding element 17 by means of the spring element 171 is elastically tensioned with respect to the support member 181. In that the spring element 171 acts in between an end 172 of the shaft 170 of the holding element 17 and the support member 181 (and hence the sensor support element 18), a tensioning force F₀ caused by the spring element 171 matches the force by which the arm 174 is biased against the sensor support element 18. In the non-actuated position of the holding element 17 of FIG. 5, hence, a balance of forces caused by the holding element 17 on the sensor device 14 is zero, the tensioning force of the spring element 171 canceling with the biasing force by which the holding arm 174 abuts the sensor support element 18.

Hence, in the non-actuated position of the holding element 17 according to FIG. 5 the holding element 17 does not cause a net force on the sensor support element 18, and thus does not, by itself, cause a deformation of the sense support element 18. A sensor reading of the sensor elements 140 of the sensor device 14 hence is zero or at least close to zero, a deformation only being caused potentially by local deformations of the sensor support element 18. The sensor reading in the non-actuated position represents a reference value S₀.

A control of the reference value S₀ may be performed so as to detect a drift on the force, which is supposed to be zero or close to zero, in the non-actuated position of the holding element 17. It is possible to electronically and automatically correct the reference value or to simply inform the user that the reference value departs from zero. Nevertheless, as it is explained further, it is not mandatory to correct the reference value to zero in order to proceed with the following sensitivity drift calibration, but the reference value may be different from zero.

In turn, when the holding element 17 is actuated and for this is displaced by a displacement X1 in the direction of motion M to remove the holding arm 174 from the sensor support element 18, as this is shown in FIG. 6, an actuation force F₁ exerted on the holding element 17 is transferred to the support member 181 via the spring element 171 and hence to the sensor support element 18. This causes a loading of the sensor device 14 in the actuated position of the holding element 17, such that the sensor device 14 outputs a sensor signal indicative of the force F₁ acting on the holding element 17.

Herein, as the spring constant of the spring element 171 is known (by measurement or by known technical parameters of the spring element 171) and as in addition in the actuated position the holding element 17 is axially displaced by a defined displacement X1, the loading force F₁ in the actuated position of the holding element 17 can be computed as

$${F}_{1} = K \cdot {X}_{1} + {F}_{0} .$$

Herein, K indicates the spring constant of the spring element 171, X₁ is the displacement by which the holding element 17 is displaced with respect to the sensor support element 18, and F₀ is an initial tensioning force caused by a tensioning of the holding element 17 with respect to the sensor support element 18 in the non-actuated position of FIG. 5.

As the sensor device 14 is loaded by a force acting onto the holding element 17 in the actuated position of the holding element 17, as shown in FIG. 6, but is in effect not loaded by the holding element 17 in the non-actuated position as shown in FIG. 5, the actuation of the holding element 17 may be used to determine a value indicative of the sensitivity of the sensor device 14.

Namely, in one embodiment the processing device 15 may be configured to determine a value indicative of the sensitivity of the sensor device 14 based on the following equation:

$${K}_{S} = ( {S}_{1} - {S}_{0} ) / {X}_{1}$$

where K_{S} indicates the value indicative of the sensitivity of the sensor device 14, S₁ indicates the sensor signal measured by the sensor device 14 in the actuated position of the holding element 17 (FIG. 6), S₀ indicates the reference value, and X₁ indicates the displacement of the holding element 17 in the actuated position.

The reference value may be determined by a sensor reading in the non-actuated position of the holding element 17 according to FIG. 5 and indicates a sensor reading in the non-loaded state of the sensor device 14. Thus, the reference value may be a value different from zero. With this force calibration, the sensitivity drift is then insensitive to any reference value offset since the sensor signal S1 is also proportionally impacted by this potential offset.

As shown in FIG. 8, the sensor output S can be assumed to linearly depend on the displacement of the holding element 17 between the non-actuated, non-loaded position (FIG. 5) and the actuated position (FIG. 6) in which the holding element 17 is displaced with respect to the non-actuated, non-loaded position by a displacement X1. The non-actuated, non-loaded position is represented by the value 0 on the X axis (to the very left of the graph), whereas the actuated position is represented by the displacement value X1. The non-actuated, non-loaded position is associated with the resting force F₀ (see FIG. 5), and the actuated position is associated with the force F₁ acting onto the holding element 17 for actuating the holding element 17 (see FIG. 6). The slope of the graph represents the value Ks indicative of the sensitivity of the sensor device 14.

In an alternative embodiment, the processing device 15 may be configured to determine a value indicative of the sensitivity of the sensor device 14 based on the following equation:

$${K}_{S}^{′} = ( {S}_{1} - {S}_{0} )$$

Actually, in a realistic setup of the infusion device 1, the processing device 15 may be configured to only determine a value K′ₛ, which is correlated to the actual sensitivity of the sensor, without taking into account the distance X1.

For instance, an initial value K′ₛ may be acquired during a calibration process during the manufacturing before releasing the infusion device 1 or during any subsequent calibration process during the lifetime of the infusion device 1. The initial value K′ₛ may then be regarded as a base value. During operation of the infusion device, then, the computation of the Ks′ value may be repeated and the actual Ks′ value may be compared to the initial base value, wherein a relative deviation of the actual Ks′ value from the initial base value indicates a change in sensitivity, allowing hence to adapt a calibration for the sensitivity.

As such, the invention allows a calibration process even without the actual need to know or to measure the distance X1.

Using the sensor sensitivity determined in this way, a drift of the sensor device 14 may be monitored. Namely, in an initial calibration procedure, for example prior to initial operation of the infusion device 1, or repeatedly at certain intervals during operation, a default value for the sensitivity of the sensor device 14 may be determined and stored in the system. During subsequent operation, then, the sensitivity of the sensor device 14 may be repeatedly determined anew, for example at every release and/or installation operation during which a piston 21 of a syringe 2 is released from the pusher device 11 or is installed on the pusher device 11. The sensitivity herein is compared to the previously stored default value, and if a (substantial) deviation of the sensitivity from the default value is observed, this may be identified as a drift in the sensor sensitivity.

If a drift is detected, countermeasures may be initiated. For example, if the deviation is substantial, but not excessive, a message may be generated advising a user that maintenance should be carried out. Such message can for example be electronically sent (via the Internet) to a maintenance service outside of the healthcare institution, for example of a manufacturer of the infusion device 1, such that the maintenance service may be ordered to perform a maintenance. If the deviation however is large, e.g. above a threshold, as the most severe countermeasure a further operation of the infusion device 1 may be prohibited, because a force measurement may no longer be reliable and hence an occlusion may not reliably be detected. In this case also a high priority alarm may be triggered.

Alternatively or in addition, the sensor sensitivity may be corrected according to current measurements of the sensitivity, such that operation may continue with a corrected sensitivity value for the sensor device 14.

The reference value as determined for calculating the sensitivity of the sensor device 14 may be determined initially during calibration, and may in addition be repeatedly determined during operation.

Herein, the reference value by itself may be used for monitoring a potential drift, in that the reference value may be initially (for example prior to actual operation) determined and stored. If subsequently, when a current reference value is determined, a deviation from the initially stored reference value is observed, this may be identified as a drift in the sensor device 14.

Referring now to FIG. 7, when a syringe 2 with its tube 20 is received in the receptacle 12 of the infusion device 1 and the piston 21 of the syringe 2 is installed on the pusher device 11, as this is shown in FIG. 7, the piston head 210 is arrested by means of the holding arm 174 of the holding element 17 on the pusher device 11, such that a movement of the pusher device 11 both in and opposite to the pushing direction A will carry the piston 21 along. Herein, by means of the spring elastic tensioning of the spring element 171 the piston head 210 is biased towards the sensor support element 18 by a force F₂, such that the piston head 210 is held in close contact to the sensor device 14, namely to the pressure transmitting element 19. The displacement of the holding element 17 in this position is noted as X2.

Herein, the tensioning force of the spring element 171 and the biasing force of the holding arm 174 on the piston head 210 match, such that a net balance of forces caused by the holding element 17 is substantially 0, similar as for the non-loaded state of the pusher device 11 according to FIG. 5. In the installed state of the piston 21 according to FIG. 7, a load reference value may be determined according to a sensor reading of the sensor device 14 in the state of FIG. 7, which subsequently may be used for example for measuring a friction force and/or an occlusion during subsequent operation of the infusion device 1.

The idea underlying the invention is not limited to the embodiments described above, but may be carried out in an entirely different fashion.

The calibration and measurement is carried out controlled by the processing device, which is programmed by software to perform corresponding routines.

It shall be noted that an actuation mechanism for actuating the holding element is not limited to the mechanism as described in EP 2 686 039 B1, but may be implemented in a different fashion.

The sensor device beneficially employs a load cell, wherein different arrangements of sensor elements may be used on the sensor device, in particular not being limited to a Wheatstone bridge. One or multiple sensor elements herein may be implemented by strain gauges or extension gauges.

### LIST OF REFERENCE NUMERALS

1 Infusion device
10 Housing
100 Front face
11 Pusher device
110 Housing
111 Mounting element
112 Membrane
12 Receptacle
13 Display device
14 Sensor device
140 Sensor element
15 Processing device
16 Storage device
17 Holding element
170 Shaft
171 Spring element
172 End
173 Actuation mechanism
174 Holding arm
18 Sensor support element
181 Support member
19 Pressure transmitting element
190 Spring element
2 Pumping device (syringe)
20 Tube
21 Piston
3 Delivery line
30, 31 End
A Pushing direction
B Patient
C1, C2 Nodes
F0 Resting force
F1 Deviation force
F2 Holding force
M Direction of motion
O Occlusion
P Pivoting direction
X1 Deviation
X2 Displacement

1. An infusion device for administering a medical fluid to a patient, comprising:
a receptacle for receiving a syringe, the syringe comprising a tube and a piston movable with respect to the tube,
a pusher device for exerting a force onto the piston for delivering a medical fluid from the tube towards a patient,
a sensor device arranged on the pusher device for measuring a force exerted on the piston by the pusher device,
a holding element arranged on the pusher device, the holding element being movable with respect to the pusher device between a non-actuated position, in which the holding element is configured to hold the piston on the pusher device, and an actuated position for installing or releasing the piston from the pusher device, and
a processing device for controlling operation of the infusion device,
wherein the processing device is configured to evaluate a sensor signal measured by the sensor device in the actuated position of the holding element and to determine a value indicative of a sensitivity of the sensor device based on said sensor signal measured by the sensor device in the actuated position of the holding element and based on a reference value.

2. The infusion device according to claim 1, wherein the sensor device comprises a sensor support element configured to react to a force exerted on the piston by the pusher device, wherein the holding element is mounted on the sensor support element and is movable with respect to the sensor support element between the non-actuated position and the actuated position.

3. The infusion device according to claim 2, wherein the sensor device comprises at least one sensor element arranged on the sensor support element and configured as a strain gauge or an extension gauge.

4. The infusion device according to claim 2, further comprising a spring element which is configured to elastically pre-tension the holding element with respect to the sensor support element along a direction of motion, wherein the spring element is elastically tensioned when moving the holding element from the non-actuated position to the actuated position.

5. The infusion device according to claim 1, wherein the processing device is configured to determine said reference value based on a sensor signal of the sensor device in the non-actuated position of the holding element.

6. The infusion device according to claim 5, wherein the processing device is configured to determine the reference value in a state of the infusion device in which no syringe is connected to the pusher device.

7. The infusion device according to claim 1, wherein the processing device is configured to determine said value indicative of the sensitivity of the sensor device based on a difference between said sensor signal measured by the sensor device in the actuated position of the holding element and the reference value.

8. The infusion device according to claim 1, wherein the processing device is configured to determine said value indicative of the sensitivity of the sensor device ) based on said sensor signal measured by the sensor device in the actuated position of the holding element and in addition based on a displacement by which the holding element is displaced with respect to the pusher device in the actuated position.

9. The infusion device according to claim 8, wherein the processing device is configured to determine said value indicative of the sensitivity of the sensor device based on the following equation:
$${K}_{S} = ( {S}_{1} - {S}_{0} ) / {X}_{1}$$
where K_{S} indicates said value indicative of the sensitivity of the sensor device, S₁ indicates the sensor signal measured by the sensor device in the actuated position of the holding element, S₀ indicates the reference value, and X₁ indicates the displacement of the holding element in the actuated position.

10. The infusion device according to claim 1, wherein the processing device is configured to determine said value indicative of the sensitivity of the sensor device based on the following equation:
$${{K}_{S}}^{′} = ( {S}_{1} - {S}_{0} )$$
where K_{S}′ indicates said value indicative of the sensitivity of the sensor device, S₁ indicates the sensor signal measured by the sensor device in the actuated position of the holding element, and S₀ indicates the reference value.

11. The infusion device according to claim 1, wherein the processing device is configured to determine a default value of the value indicative of the sensitivity of the sensor device in a calibration procedure.

12. The infusion device according to claim 11, wherein the processing device is configured to identify a drift in the sensitivity of the sensor device based on a comparison of the default value to a value indicative of the sensitivity of the sensor device determined subsequent to the calibration procedure.

13. The infusion device according to claim 1, wherein the holding element is configured, in an operative state in which the holding element is in the non-actuated position and operatively connects the piston to the pusher device, to bias the piston into abutment with the sensor device.

14. The infusion device according to claim 13, wherein the processing device is configured to determine a load reference value based on a sensor signal of the sensor device in said operative state.

15. A method for operating an infusion device for administering a medical fluid to a patient, comprising:
receiving a syringe in a receptacle of the infusion device, the syringe comprising a tube and a piston movable with respect to the tube,
exerting, using a pusher device, a force onto the piston for delivering a medical fluid from the tube towards a patient,
measuring, using a sensor device arranged on the pusher device, a force exerted on the piston by the pusher device,
controlling operation of the infusion device using a processing device,
evaluating, using the processing device, a sensor signal measured by the sensor device in an actuated position of a holding element, the holding element being arranged on the pusher device and being movable with respect to the pusher device between a non-actuated position, in which the holding element is configured to hold the piston on the pusher device, and the actuated position for releasing the piston from the pusher device, and
determining, using the processing device, a value indicative of a sensitivity of the sensor device based on said sensor signal measured by the sensor device in the actuated position of the holding element and based on a reference value.
